(19) United States
(12) Patent Application Publication
(10) Pub. No.: US 2022/0409916 A1
(43) Pub. Date: Dec. 29, 2022

(54) DEVICE FOR SUPPORTING HEIGHT GROWTH BY USING PULSED MICRO-ELECTROMAGNETIC FIELD, AND METHOD FOR DRIVING DEVICE

(71) Applicant: MN CORPORATION, Seoul (KR)

(72) Inventor: Kyoung-Soo MIN, Seoul (KR)

(21) Appl. No.: 17/778,144

(22) PCT Filed: Apr. 28, 2020
(86) PCT No.: PCT/KR2020/005565

§ 371 Date: May 19, 2022

(30) Foreign Application Priority Data
Jan. 2, 2020 (KR) 10-2020-0000189

(51) Int. Cl. A61N 2/02; H03F 3/189; H01Q 1/27

(52) U.S. Cl. CPC A61N 2/02; H03F 3/189; H01Q 1/273

(57) ABSTRACT

Proposed are a device for supporting height growth by using a pulsed micro-electromagnetic field, and a method for driving the device. According to an embodiment, the device may include an antenna band part including a first antenna and a second antenna which are provided inside a covering part that is configured to surround a joint and which are configured to generate a micro-magnetic field at the joint in a contactless manner, and may include a control unit configured to generate the micro-magnetic field by forming a high frequency voltage and providing the high frequency voltage to the antenna band part, the control unit being configured to adjust the formed high frequency voltage, on the basis of a sensing value of a micro-electromagnetic field sensed at the second antenna, such that a micro-magnetic field corresponding to a diameter of the joint is provided.

### TECHNICAL FIELD

The present disclosure relates to a device for supporting height growth by using a pulsed micro-electromagnetic field, and to a method for driving the device. More particularly, the present disclosure relates to a device for supporting height growth by using a pulsed micro-electromagnetic field, the device promoting growth by generating a micro-electromagnetic field at children's growth plates as an example in a contactless manner in which an electrode that is in direct contact with the skin does not exist, and relates to a method for driving the device.

### BACKGROUND ART

Cartilage of a growth plate consists of cartilage cells. When stimulation is applied to the cartilage cells through exercise, growth of a length of a bone occurs through division and proliferation of the cartilage cells. As an example of a growth process of a leg, in growth of a length of a femur of the leg, division and proliferation of the cartilage cells occurs actively in a center portion of an epiphyseal cartilage at first. Then, among the divided and proliferated cartilage cells, the cartilage cells including a connective tissue turn into an osteoblast, and a bone is formed.

Conventionally, as a method of supporting height growth, methods such as physically massaging growth plates, stimulating acupuncture points on soles to promote growth hormone secretion, and so on have been proposed.

However, in the method of physically massaging growth plates, when an intensity of pressure and an intensity of thermal stimulation in a method using a combination of external massage with a motor or air pressure and thermal stimulation are wrongly adjusted, there is a disadvantage that growth plates may be damaged.

In addition, in the method of stimulating acupuncture points on soles, socks are required to be removed and an electrical stimulation is required to be directly applied to the soles of the feet. However, generally, use of a device for electrically stimulating the skin is not recommended internationally for children and adolescents. Further, there is an inconvenience that the device is difficult to use in daily life and can be only used while sleeping since the device is required to be attached to the soles of the feet.

### DISCLOSURE

### Technical Problem

Accordingly, the present disclosure has been made keeping in mind the above problems occurring in the related art, and an objective of an embodiment of the present disclosure is to provide a device for supporting height growth by using a pulsed micro-electromagnetic field, the device promoting growth by generating a micro-electromagnetic field at children's growth plates as an example in a contactless manner in which an electrode that is in direct contact with the skin does not exist, and to a method for driving the device.

### Technical Solution

According to an embodiment of the present disclosure, there is provided a device for supporting height growth by using a pulsed micro-electromagnetic field, the device including: an antenna band part including a first antenna and a second antenna which are provided inside a covering part that is configured to surround a joint and which are configured to generate a micro-magnetic field at the joint in a contactless manner; and a control unit configured to generate the micro-magnetic field by forming a high frequency voltage and providing the high frequency voltage to the antenna band part, the control unit being configured to adjust the formed high frequency voltage, on the basis of a sensing value of a micro-electromagnetic field sensed at the second antenna, such that a micro-magnetic field corresponding to a diameter of the joint is provided.

The first antenna and the second antenna of the antenna band part may be respectively formed in a non-linear shape, and may be spaced apart from each other.

The control unit may include: a high frequency forming part configured to form the high frequency voltage; and an output adjusting part configured to adjust the formed high frequency voltage and output the adjusted high frequency voltage to the antenna band part.

The control unit may include: a sensing part configured to sense a micro-magnetic field output to the antenna band part for a predetermined time; an amplifying part configured to amplify a sensing value of the sensed micro-magnetic field; and an RC filter part configured to convert the amplified sensing value into a DC level signal so as to adjust the high frequency voltage.

The control unit may be configured to calculate a ratio of a reference area related to the diameter of the joint on the basis of the converted DC level signal, and may be configured to adjust the high frequency voltage on the basis of the calculated ratio.

The control unit may be configured to preset a reference output value that corresponds to a median value for a diameter size of the joint, and may be configured to adjust the high frequency voltage on the basis of the preset reference output value and the calculated ratio.

The control unit may be configured to modify a pulse width of the high frequency voltage, thereby generating a constant micro-magnetic field regardless of a size of the joint.

The control unit may be configured to set the reference output value with a duty of 50%, and may be configured to reduce the pulse width to be smaller than the 50% duty when a sensing value amplified by the amplifying part is larger than the reference output value and may be configured to increase the pulse width to be larger than the 50% duty when the sensing value amplified by the amplifying part is smaller than the reference output value, thereby generating the constant micro-magnetic field.

In addition, according to an embodiment of the present disclosure, there is provided a method for driving a device for supporting height growth by using a pulsed micro-electromagnetic field, the method including: generating, by an antenna part, a micro-magnetic field at a joint in a contactless manner by a first antenna and a second antenna that are provided inside a covering part which surrounds the joint; and forming, by a control unit, a high frequency voltage and providing the high frequency voltage to the antenna band part and thus generating the micro-magnetic field, and adjusting the generated micro-magnetic field, on the basis of a sensing value of a micro-magnetic field sensed at the second antenna, such that a micro-magnetic field corresponding to a diameter of the joint is provided.

The first antenna and the second antenna of the antenna band part may be respectively formed in a non-linear shape, and may be spaced apart from each other.

The method for driving the device may further include: forming, by a high frequency forming part, the high frequency voltage; and outputting, by an output adjusting part, performed by adjusting the formed high frequency voltage and outputting the adjusted high frequency voltage to the antenna band part.

The method for driving the device may further include: sensing, by a sensing part, a micro-magnetic field output to the antenna band part for a predetermined time; amplifying, by an amplifying part, a sensing value of the sensed micro-magnetic field; and converting, by an RC filter part, the amplified sensing value into a DC level signal so as to adjust the high frequency voltage.

The method for driving the device may further include: calculating a ratio of a reference area related to the diameter of the joint on the basis of the converted DC level signal; and adjusting the high frequency voltage on the basis of the calculated ratio.

The adjusting of the high frequency voltage may include: presetting a reference output value that corresponds to a median value for a diameter size of the joint; and adjusting the high frequency voltage on the basis of the preset reference output value and the calculated ratio.

In the adjusting of the high frequency voltage, a pulse width of the high frequency voltage may be modified, thereby generating a constant micro-magnetic field regardless of a size of the joint.

In the adjusting of the high frequency voltage, the reference output value may be set with a duty of 50%, and the pulse width may be reduced to be smaller than the 50% duty when a sensing value amplified by the amplifying part is larger than the reference output value and the pulse width may be increased to be larger than the 50% duty when the sensing value amplified by the amplifying part is smaller than the reference output value, thereby generating the constant micro-magnetic field.

### Advantageous Effects

According to an embodiment of the present disclosure, physical pressure, thermal stimulation, or electrical stimulation in a skin-contact manner is not required, so that children and adolescents can use the device of the present disclosure safely and comfortably in a contactless manner even while wearing clothes.

### DESCRIPTION OF DRAWINGS

FIG. 1 is a view illustrating a device for supporting height growth according to an embodiment of the present disclosure,

FIG. 2 is an exploded perspective illustrating the device for supporting height growth in FIG. 1,

FIG. 3 is a view illustrating a principle of promoting height growth,

FIG. 4 is a block diagram illustrating a driving mechanism of the device for supporting height growth in FIG. 1,

FIG. 5 is a block diagram illustrating another driving mechanism of the device for supporting height growth in FIG. 1,

FIG. 6 is a flowchart illustrating a process of driving the device for supporting height growth in FIG. 1,

FIG. 7 is a flowchart illustrating a process of adjusting an output strength of a pulse of the device for supporting height growth in FIG. 1, and

FIG. 8 is a flowchart illustrating a process of driving the device for supporting height growth in FIG. 1 according to another embodiment.

### BEST MODE

Hereinafter, an embodiment of the present disclosure will be described in detail with reference to the accompanying drawings.

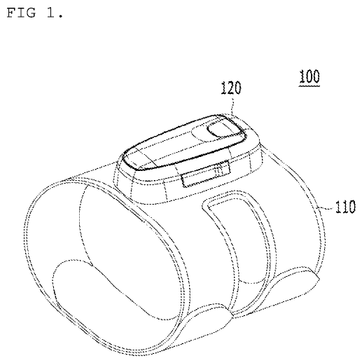

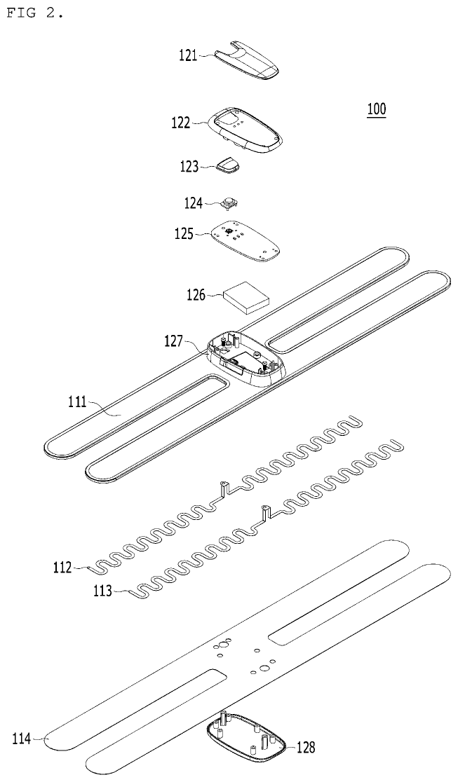

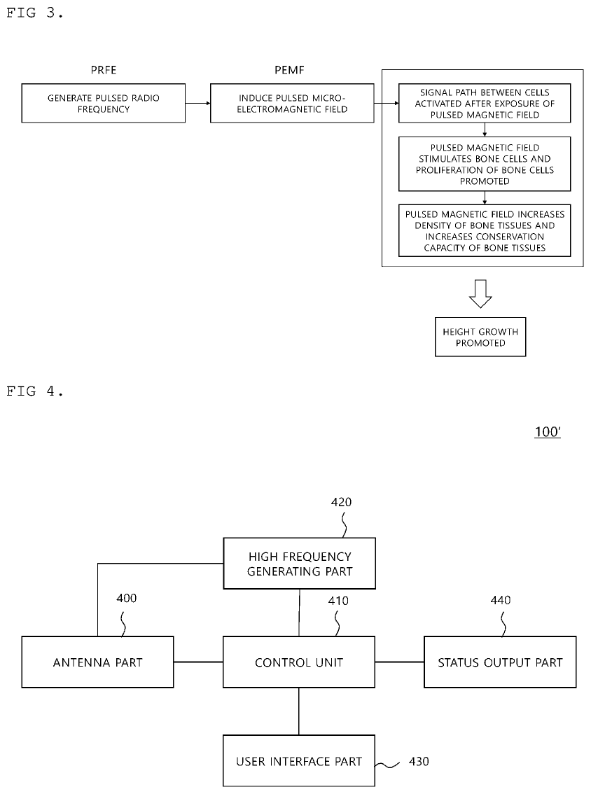

FIG. 1 is a view illustrating a device for supporting height growth according to an embodiment of the present disclosure, FIG. 2 is an exploded perspective illustrating the device for supporting height growth in FIG. 1, and FIG. 3 is a view illustrating a principle of promoting height growth.

As illustrated in FIGS. 1 and 2, a device 100 for supporting height growth by using a pulsed micro-electromagnetic field (hereinafter, referred to as a device for supporting height growth) according to an embodiment of the present disclosure includes some or all of an antenna band part 110 and a control unit 120 (or body part).

Here, the term “including some or all” means that the control unit 120 is integrated into the antenna band part 110, is formed integrally with the antenna band part 110, or the like, and it is exemplified in the following description that all of them are included to help sufficiently understand the present disclosure.

As illustrated in FIG. 2, antenna parts 112 and 113 (a first antenna 112 and a second antenna 113) for inducing a pulsed micro-electromagnetic field to a human body are mounted in the antenna band part 110, and the antenna band part 110 is formed in a band shape for attaching the antenna band part 110 to a joint. For example, the antenna band part 110 is capable of performing 360-degree care in which a knee or an elbow of children in growth period is surrounded with the antenna band part 110. Growth plates are positioned above and below the knee. Therefore, the antenna band part 110 is configured to fully surround the growth plates such that a micro-magnetic field antenna is evenly operated. For example, the antenna band part 110 has a dual band structure that surrounds both a growth plate of an upper bone of the joint and a growth plate of a lower bone of the joint.

As illustrated in FIGS. 1 and 2, the antenna band part 110 includes the antenna parts 112 and 113 and covering parts 111 and 114 (a band upper plate 111 and a band lower plate 114). Further, the antenna parts 112 and 113 include the first antenna 112 and the second antenna 113 that are formed in an S shape which is a non-linear shape as an example. The first antenna 112 and the second antenna 113 may be formed of various conductive materials, and may be formed in various shapes such as an S shape and the like. Of course, when the first antenna 112 and the second antenna 113 surround the growth plates, the first antenna 112 and the second antenna 113 form a circular loop antenna. A strength of a (electro) magnetic field applied to the human body may vary according to a material and a shape of the first and second antennas 112 and 113, so that a high frequency (or a high frequency voltage or a high frequency signal) may be adjusted in consideration of this. Here, when the high frequency means that a frequency of a predetermined level voltage is high, a high voltage means that a level of a predetermined voltage is high. Therefore, in an embodiment of the present disclosure, various aspects may be considered. In other words, in the present specification, a high frequency signal having a low voltage also can be used. As illustrated in FIG. 2, the covering parts 111 and 114 may include the band upper plate 111 and the band lower plate 114.

In the control unit (or body part) 120, a driving part such as a PCB substrate and so on is included inside an outer casing (or body frame) that forms an outer periphery of the control unit (or body part) 120. More specifically, the control unit 120 may include a window 121, a top cover 122, an operation button 123, a silicon button 124, a circuit substrate 125, a rechargeable battery 126, a bottom cover 127, and an under-cover 128. In other words, in the control unit 120, the bottom cover 127 is coupled to the under-cover 1280 with the antenna band part 110 interposed therebetween. The under-cover 128 may be brought into contact with a user's skin or clothes. Of course, a structure of the control unit 120 may be variously changed. Above all, in an embodiment of the present disclosure, a separate electrode that is in contact with the skin is not used. That is, the pulsed micro-electromagnetic field is applied to the growth plates in a contactless manner.

The control unit 120 includes an indicator LED, and a power/mode button. Further, the bottom cover 127 include the rechargeable battery 126. In this regard, the bottom cover 127 may be referred to as a charging terminal cover. When the user selects a sleep (or sleep)/a normal/a focus mode, the indicator LED indicates each mode by an LED. The power/mode button turns a power on and off, and selects a mode. In addition, the charging terminal cover, which is the bottom cover 127, is a cover protecting a terminal that connects a micro USB charger.

In addition, the control unit 120 according to an embodiment of the present disclosure may further include a vibration motor. By transmitting a feedback from the vibration motor to the user, the user may feel a sense of use. Of course, in an embodiment of the present disclosure, three care modes such as the sleep mode, the normal mode, and the focus mode can be operated. Further, in the sleep mode, a vibration feedback does not exist, and can be operated for about eight hours. Furthermore, the normal mode can be operated for about four hours, and the focus mode can be operated for about two hours. In the normal mode and the focus mode, the feedback is transmitted to the user by using the vibration motor, and the user may sense the normal mode and the focus mode through the feedback of the vibration motor. After the rechargeable battery 126 is fully charged, the normal mode can be used for five days to six days on the basis of once a day use. After the antenna band part 110 is worn on the knee or the elbow, the device 100 can be operated by pressing the button. After use, the power may be turned off automatically. In other words, after the device 100 is operated in the corresponding mode, the power may be automatically turned off when a predetermined time elapses.

Referring to the principle of promoting height growth with reference to FIG. 3, the control unit 120 generates a pulsed high frequency on the basis of a user's command, and a Pulsed Electro-Magnetic Field (PEMF) therapy method using a Pulsed Radio Frequency Energy (PRFE) is applied. The PEMF therapy method, which generates an electromagnetic field and is used for performing a treatment and for promoting health, has been widely used in a hospital and a home care for the past 40 years, mainly in the United States. There are several methods of generating such a PEMF. Among them, an embodiment of the present disclosure employs a method of generating a micro-electromagnetic field through a pulsed high frequency method. Further, as another example of a PEMF generation method, there is a Direct Current (DC) pulse method. The high frequency employed in the present disclosure is 40.68 MHz. Further, a device that generates a radio wave other than commercially licensed normal frequencies is required to obtain a radio frequency use license separately. Furthermore, internationally, a range of a frequency band of the Industry Science and Medical (ISM) band designated by the United States Federal Communications Commission (FCC) can be used without a license. In addition, for medical devices, frequencies of 13.56 MHz, 27.12 MHz, and 40.68 MHz are mainly used for treatment, and an embodiment of the present disclosure also employs the frequency of 40.68 MHz. However, the frequency employed in an embodiment of the present disclosure is not particularly limited to any one frequency.

In the frequencies used in medical use, the frequency of 40.68 MHz is located at the highest frequency band compared to the frequencies of 13.56 MHz or 27.12 MHz, so that the frequency of 40.68 MHz has advantages that a cell activation proceeds faster than other frequencies and the amount of energy transmitted to the human body is larger than other frequencies when the frequency of 40.68 MHz is induced to the human body. In addition, the relationship between frequency and antenna length is inversely proportional to each other. Therefore, in order to realize efficient radio wave transmission, the smaller the frequency, the longer the antenna is designed, and the larger the frequency, the shorter the antenna is designed. Therefore, in the frequency of 40.68 MHz, a length of the antenna can be designed relatively short compared to a length of an antenna of other licensed medical frequencies, so that a pulsed electromagnetic field can be more efficiently transmitted to the human body.

Through this, in the device 100 for supporting height growth according to an embodiment of the present disclosure, after a bone is exposed to the pulsed electromagnetic field, height growth of children and adolescents in a growing period are promoted through a cell activation of the bone and an energy supply to the bone up to a molecular level cells in the bone and up to a cellular and tissue level cells in the bone. Since a magnetic field is generated where electricity is generated, high-purity micro-magnetic field energy that vibrates 4,068,000 times per second is generated. In other words, the device 100 for supporting height growth activates a signal path between cells, promotes proliferation of bone cells since the bone cells are stimulated by the pulsed magnetic field, and increases a conservation capability of a density of bone tissues by using the pulsed magnetic field, so that height growth is promoted. In addition, the device 100 for supporting height growth also alleviates growing pains. The device 100 for supporting height growth alleviates a joint pain in the growing period, the joint pain being experienced by more than 30% of all children. Further, the device 100 for supporting height growth improves blood flow in a joint region and alleviates a pain in the joint region by using the pulsed micro-magnetic field.

FIG. 4 is a block diagram illustrating a driving mechanism of the device for supporting height growth in FIG. 1.

As illustrated in FIG. 4, a device 100′ for supporting height growth in FIG. 1 according to another embodiment of the present disclosure may include some or all of an antenna part 400, a control unit 410, a high frequency generating part 420, a user interface part 430, and a status output part 440.

Here, the term “including some or all” means that the device 100′ for supporting height growth can be configured such that some components such as the status output part 440 is omitted, or that the device 100′ for supporting height growth can be configured such that some components such as the high frequency generating part 420 is integrated with another component such as the control unit 410. Further, it is exemplified in the following description that all of them are included to help sufficiently understand the present disclosure.

The antenna part 400 may include a first antenna and a second antenna that are spaced apart from each other at a predetermined distance. Here, it is preferable that positions of the first antenna and the second antenna are respectively set to correspond to positions of the growth plate of the upper and lower bones when the device 100′ for supporting height growth is mounted on the joint. The first antenna and the second antenna are formed in a non-linear shape such as a S shape, but lengths of the first antenna and the second antenna may be determined according to a predetermined high frequency pulse (for example, 40.68 MHz). The predetermined high frequency pulse is a carrier frequency, and may be formed by converting a pulse frequency of 9 Hz to 33 Hz. The pulse frequency of 9 Hz to 33 Hz may be generated by an oscillator.

The control unit 410 may serve to control overall operations of the antenna part 400, the high frequency generating part 420, the user interface part 430, and the status output part 440. For example, the control unit 410 is configured to control the high frequency generating part 420 on the basis of the user's command input to the user interface part 430, so that the electromagnetic field is generated according to the user's command. For example, the device 100′ for supporting height growth may be operated in three different care modes that are a sleep mode, a normal mode, and a focus mode. Therefore, when the user selects the sleep mode through the user interface part 430, the high frequency generating part 420 is controlled accordingly. Particularly, in the sleep mode, the control unit 410 controls the status output part 440 such that a motor is not operated and a vibration is not generated. On the other hand, in the normal mode and the focus mode, a high frequency, i.e., an electromagnetic wave, corresponding to each mode is generated and is provided to the antenna part 400, and the motor of the status output part 440 is controlled such that the vibration corresponding to each mode is generated. In the focus mode, the user may feel a large vibration. Here, selecting a mode can be seen as a part of a process of generating the pulsed micro-electromagnetic field that corresponds to each mode by using the high frequency generating part 420 when the user's command is input.

In addition, the control unit 410 may include a high frequency sensing part. Of course, the high frequency sensing part may be included in the antenna part 400 or the high frequency generating part 420, so that another embodiment of the present disclosure is not specifically limited to any one form. The control unit 410 senses the high frequency provided to the antenna part 400 through the high frequency sensing part, and may control a vibration motor or an indicator LED on the basis of a sensing value. Further, the sensing value can be used to feedback control the high frequency generating part 420. For example, a pulse width modulation can be performed such that a constant micro-electromagnetic field is provided in a designated mode, regardless of a body size of children in the growing period. The pulse width modulation is performed by adjusting a duty ratio.

The high frequency generating part 420 may include a high frequency forming part that forms a pulse voltage of a predetermined frequency, and a frequency adjusting part that adjust a frequency of the generated pulse voltage. For example, according to another embodiment of the present disclosure, the high frequency forming part may output a high frequency pulse voltage of 40.68 MHz through the oscillator, and the frequency adjusting part adjusts an output power by changing a duty through the pulse width modulation. Of course, the high frequency generating part 420 according to another embodiment of the present disclosure may be turned on and turned off by the control unit 410. In other words, when a power input operation from the user interface part 430 is performed, the control unit 410 may operate the high frequency generating part 420 such that the high frequency is generated according to a request of the user.

The user interface part 430 receives the user's command, i.e. a control command, from the user. The user interface part 430 may include a button or the like for operating the device 100′ for supporting height growth, or may include a voice receiving part, such as a microphone, for operating the device 100′ for supporting height growth through a voice command. The received command of the user is transmitted to the control unit 410, and the control unit 410 may control the high frequency generating part 420 on the basis of the received command of the user. Of course, when the control command for operating the device 100′ for supporting height growth in the normal mode or the focus mode is received, the control unit 410 may control the vibration motor of the status output part 440 to be operated correspondingly. For example, the vibration of the vibration motor in the focus mode may be more intensive than the vibration of the vibration motor in the normal mode.

The status output part 440 may include a light-emitting part and a vibrating part. The light-emitting part includes a light-emitting element such as a LED, and may display a current operation status of the device 100′ for supporting height growth. For example, when the device 100′ for supporting height growth is operating in the sleep mode, the LED displaying the sleep mode can emit light. In addition, the vibrating part includes the vibration motor. Further, the vibration motor maintains the turned off state in the sleep mode, but may generate the corresponding vibration in the normal mode or in the focus mode.

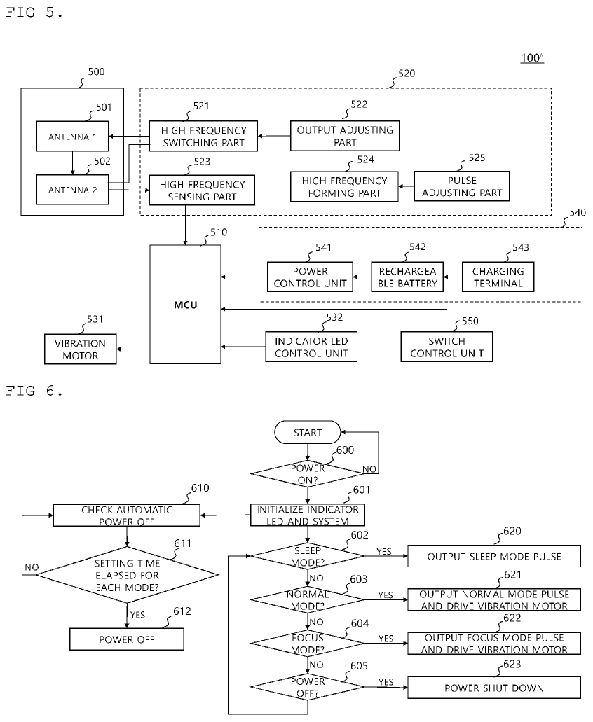

FIG. 5 is a block diagram illustrating another driving mechanism of the device for supporting height growth in FIG. 1.

As illustrated in FIG. 5, a device 100″ for supporting height growth according to still another embodiment of the present disclosure may include some or all of an antenna part 500, a control unit 510, a high frequency generating part 520, a status output part 531 and 532 (a vibration motor part 531 and an indicator LED control unit 532), a power supply part 540, and a switch control unit 550. Here, the term “including some or all” does not significantly differ from the meaning described above.

The antenna part 500 includes a first antenna part 501 and a second antenna part 502. Further, a first antenna of the first antenna part 501 and a second antenna of the second antenna part 502 may be spaced apart from each other at a predetermined distance, and may be formed in a shape in which the growth plates of the upper and lower bones of the joint are surrounded.

The control unit 510 may serve to control overall operations of the high frequency generating part 520, the status output parts 531 and 532, the power supply part 540, and so on that are configuring the device 100″ for supporting height growth in FIG. 5. For example, when a power button is pressed through the switch control unit 550, the control unit 510 may receive a power by controlling a power control unit 541 and may supply the power to the high frequency generating part 520, the status output parts 531 and 532, and so on.

The high frequency generating part 520 may include some or all of a high frequency switching part 521, an output adjusting part 522, a high frequency sensing part 523, a high frequency forming part 524, and a pulse control unit 525. The high frequency switching part 521 serves to select the output or sensing of an antenna 2 (or the second antenna). Further, the high frequency switching part 521 switches the antenna 2 to the sensing during an initial high frequency sensing process (for example, three seconds), and switches the antenna 2 to be connected to an output part when sensing and output control are completed. The output adjusting part 522 supplies the high frequency pulse generated from the high frequency forming part 524 to an antenna 1, and the high frequency pulse adjusts an output power by converting a duty through the pulse width modulation. The antenna 1 is mounted inside a band that is attached to the elbow or the knee joint. The high frequency sensing part 523 is a part that senses the strength of the electromagnetic field from the antenna 2, the electromagnetic field being formed by the high frequency which is output through the high frequency switching part 521 and which is then passing through the antenna 1. Further, an amplifier (or an amplifying part) that amplifies a high frequency input signal is mounted in the high frequency sensing part 523. More accurately, an amplifier (or an amplifying part) that senses and amplifies a high frequency input signal is mounted in the high frequency sensing part 523. An RF amplifier may be used as the amplifier. An operational amplifier that has a designated gain A may be used as the amplifier. Here, a gain may refer to an amplification factor. The high frequency forming part 524 may include an oscillator having an oscillation frequency of 40.68 MHz, and an output power is adjusted by changing a duty by performing the pulse width modulation through the output adjusting part 522. The pulse control unit 525 controls an on/off state of the high frequency forming part 524, and controls the high frequency pulse to be output in a burst form. A pulse burst refers to a finite number of continuous pulses.

the status output parts 531 and 532 include the vibration motor part 531 and the indicator LED control unit 532. Even if the output pulsed micro high frequency is transmitted to the human body through an antenna band, there is no feeling in the skin or bones. Therefore, a vibration motor is mounted, and a sense of vibration is applied to the user in the normal mode and the focus mode except for the sleep mode, so that the user feels the feedback feeling according to a pulse transmission. In addition, there are three indicator LEDs that respectively indicate the sleep mode, the normal mode, and the focus mode according to the selection.

The power supply part 540 includes the power control unit 541, a rechargeable battery 542, and a charging terminal 543. A power of the rechargeable battery 542 is supplied by a lithium polymer rechargeable battery having a capacity of 7.4 V and 500 mAh. Further, for example, when a power button is selected, the power is supplied and controlled by a FET element of the power control unit 541.

The switch control unit 550 may include various buttons for receiving the user's command. Typically, the power button or buttons for operating operation modes such as the sleep mode, the normal mode, and so on may be included. In addition, various components such as the voice receiving part for receiving the voice command may be included.

In addition to the description as described above, the antenna part 500, the control unit 510, the high frequency generating part 520, the status output parts 531 and 532, the power supply part 540, and the switch control unit 550 that are in FIG. 5 may perform various operations, and the description thereof has been sufficiently described above, so that the detailed description thereof will be replaced with the description described above.

FIG. 6 is a flowchart illustrating a process of driving the device for supporting height growth in FIG. 1.

Referring to FIG. 6 with FIG. 1 for convenience of the description, when a power of the device 100 for supporting height growth according to an embodiment of the present disclosure turned on, the indicator LED and a system are initialized (S600 and S601). For example, when the power is turned on by pressing the power button, the LED of the normal mode is turned on and the normal mode is operated by default. Otherwise, the device 100 for supporting height growth may apply the high frequency at the predetermined level to the human body for the predetermined time (for example, three seconds) and may senses the high frequency at the predetermined level, and then may perform the pulse width modulation for adjusting the duty of the high frequency such that the constant micro-magnetic field is output regardless of the body size of the children.

In addition, when the user presses the button, the device 100 for supporting height growth determines whether the normal mode or the focus mode is selected or not (S602 to S605). Of course, in the normal mode, when there is a power off request, the power may be shut down (S623). When the user presses the button at least two seconds even while the device 100 is in use, the power may be turned off.

If the device 100 for supporting height growth is in the sleep mode, the device 100 output a sleep mode pulse (S620). In the sleep mode operation, the vibration motor is not driven.

In addition, in the normal mode, the device 100 for supporting height growth outputs a normal mode pulse, and drives the vibration motor (S603 and S621). The vibration generated by the vibration motor in the normal mode may be weaker than the vibration in the focus mode.

Further, in the focus mode, the device 100 for supporting height growth outputs a focus mode pulse, and may drive the vibration motor corresponding to the focus mode (S604 and S622). For example, the vibrations of the vibration motor can be differentiated by increasing the number of control signals (or driving voltages) provided to the vibration motor.

In addition, after the system is turned on, the device 100 for supporting height growth checks an automatic power off function (S610). For example, the device 100 for supporting height growth may check the remaining battery level. For example, the device 100 for supporting height growth may determine whether the set time for each mode has elapsed (S611) and may automatically turn off the power when the set time has elapsed (S612). For example, the device 100 for supporting height growth operates for eight hours in the sleep mode, four hours in the normal mode, and two hours in the focus mode, and the power of the device 100 for supporting height growth may be automatically turned off after the set time has elapsed. A timer may be used to check the time. The timer can be hardware or software.

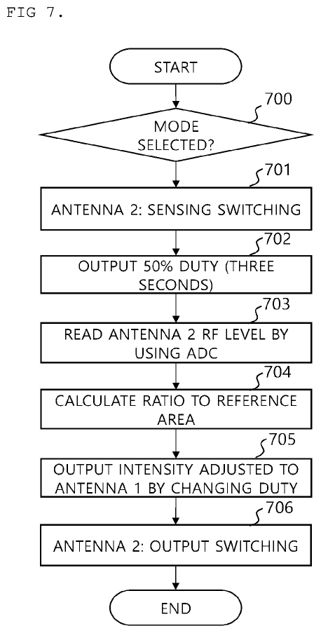

FIG. 7 is a flowchart illustrating a process of adjusting an output strength of a pulse of the device for supporting height growth in FIG. 1.

Referring to FIG. 7 with FIG. 1 for convenience of the description, the device 100 for supporting height growth according to an embodiment of the present disclosure may perform the sensing and the feedback using the antenna 2 as an example.

In the device 100 for supporting height growth, since the antenna 1 is connected such that the antenna 1 always output a micro-magnetic field when a mode is selected by a switch, the antenna 2 is switched such that the antenna 2 is connected to a sensing part so as to sense an output strength of the micro-magnetic field of the antenna 1 (S700 and S701).

In addition, in a state in which the antenna 1 outputs the micro-magnetic field and the antenna 2 is connected to the sensing part, the device 100 for supporting height growth outputs a micro-magnetic field having the duty of 50% for three seconds (S702).

Then, in the device 100 for supporting height growth, the radio wave of 40.68 MHz received for three seconds from the antenna 2 has a signal amplified through the RF amplifier, the signal is converted into a DC level signal through an RC filter, and the device 100 reads a level of the DC level by using an Analog-Digital Converter (ADC) of a control unit (for example, an MCU) (S703). Here, the RC filter may include a resistor R and a capacitor C, and may convert an RF amplified signal to a direct current by acting as a smoothing circuit.

The strength of output is expressed in mW/cm², which is mW per unit area. The band-shaped antenna is mounted on an elbow or a knee. Further, since a diameter of the elbow or the knee of children or adolescents who are six to eighteen years old and who use the device 100 for supporting height growth is varied, a diameter of the circular loop antenna that is formed by the mounted band may be distributed in a wide range of about 5 cm to 15 cm according to the size of the diameter of the band when the band is mounted on the elbow or the knee.

Therefore, in the device 100 for supporting height growth, a ratio of the diameter to a reference area is calculated and the duty is changed according to the calculation result so that the micro-magnetic field in which the strength is adjusted is output from the antenna 2 (or the antenna 1) (S705 and S706).

For example, in the device 100 for supporting height growth, 10 cm, which is a median value of 5 cm to 15 cm, is set as a 0.1 mW/cm² that is a reference output value, and the reference output value is configured to be output when the duty is 50%. Therefore, when an RF-amplified value received via the antenna 2 is higher than the reference output value, the duty is reduced to less than 50%, and when the RF-amplified value received via the antenna 2 is lower than the reference output value, the duty is increased to more than 50%, so that the constant output of the micro-magnetic field can be realized regardless of the body size.

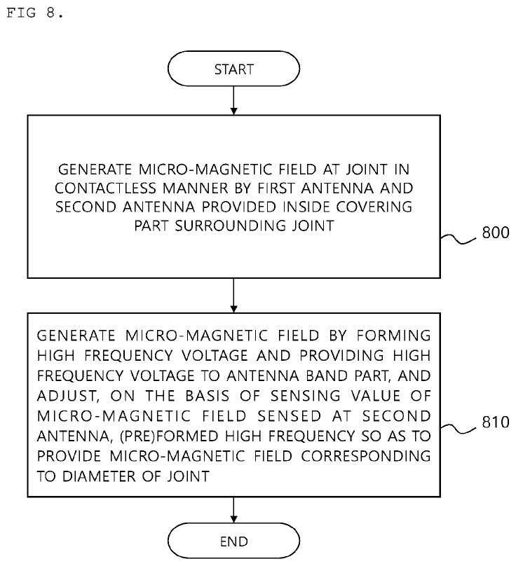

FIG. 8 is a flowchart illustrating a process of driving the device for supporting height growth in FIG. 1 according to another embodiment.

Referring to FIG. 8 with FIG. 1 for convenience of the description, the device 100 for supporting height growth in FIG. 1 according to an embodiment of the present disclosure generates a micro-magnetic field at a joint in a contactless manner by a first antenna and a second antenna that are provided inside a covering part of the antenna band part 110 surrounding the joint (S800). For example, when a power is applied, the device 100 for supporting height growth senses such as a body size of children in a growing period and a diameter of the joint, and may generate a micro-magnetic field having the reference output value for a predetermined time so as to provide a constant micro-magnetic field. In addition, the constant micro-magnetic field can be provided by adjusting the reference output value on the basis of a sensing value.

The device 100 for supporting height growth generates a micro-magnetic field by generating a high frequency voltage (or a signal) and transmitting the high frequency voltage to the antenna band part 110, and may adjust the pre-generated high frequency voltage so as to provide a micro-magnetic field corresponding to a diameter of the joint, on the basis of the sensing value of the micro-magnetic field that is sensed at the second antenna (S810). For example, the device 100 for supporting height growth adjust the high frequency voltage such that the micro-magnetic field having the constant size is provided regardless of the body size of children. To this end, the pulse width modulation control in which the duty is adjusted may be performed. For example, at a value having the same frequency, when a duty-on time is adjusted, a time or amount for which the micro-magnetic field is applied increases. For example, when a total duty time is set to 100 and it is assumed that 50% of the total duty time applies the micro-magnetic field and the remaining 50% of the total duty time does not apply the micro-magnetic field, the ratio of the applied micro-magnetic field and the non-applied micro-magnetic field may be adjusted to 60:40 by adjusting the duty ratio.

Other than the above description, the device 100 for supporting height growth can perform various operations, and other detailed contents have been sufficiently described above and thus are omitted herein.

Although all elements constituting the embodiments are described as integrated into a single one or to be operated as a single one, the present exemplary embodiment is not necessarily limited thereto. According to embodiments, all of the elements may be selectively integrated into one or more and be operated as one or more within the objective and the scope of the present disclosure. Each of the elements may be implemented as independent hardware. Alternatively, some or all of the elements may be selectively combined into a computer program having a program module performing some or all functions combined in one or more pieces of hardware. A plurality of codes and code segments constituting the computer program may be easily understood by those skilled in the art to which the present disclosure pertains. The computer program may be stored in non-transitory computer readable media such that the computer program is read and executed by a computer to implement embodiments of the present disclosure.

The non-transitory computer readable medium is a medium that semi-permanently stores data and from which data is readable by a device, but not a medium that stores data for a short time, such as register, a cache, a memory, and the like. In detail, the aforementioned various applications or programs may be stored in the non-transitory computer readable medium, for example, a compact disc (CD), a digital versatile disc (DVD), a hard disc, a Blu-ray disc, a universal serial bus (USB), a memory card, a read only memory (ROM), and the like, and may be provided.

The foregoing exemplary embodiments and advantages are merely exemplary and are not to be construed as limiting the present inventive concept. The exemplary embodiments can be readily applied to other types of apparatuses. Also, the description of the exemplary embodiments is intended to be illustrative, and not to limit the scope of the claims, and many alternatives, modifications, and variations will be apparent to those skilled in the art.

### DESCRIPTION OF REFERENCE NUMERALS

100, 100′, 100″: device for supporting height growth

112: first antenna 113: second antenna

120: control unit 400, 500: antenna part

410, 510: control unit 420, 520: high frequency generating part

430: user interface part 440: status output part

531: vibration motor part 532: indicator LED control unit

540: power supply part 550: switch control unit

1. A device for supporting height growth by using a pulsed micro-electromagnetic field, the device comprising:
an antenna band part comprising a first antenna and a second antenna which are provided inside a covering part that is configured to surround a joint and which are configured to generate a micro-magnetic field at the joint in a contactless manner; and
a control unit configured to generate the micro-magnetic field by forming a high frequency voltage and providing the high frequency voltage to the antenna band part, the control unit being configured to adjust the formed high frequency voltage, on the basis of a sensing value of a micro-electromagnetic field sensed at the second antenna, such that a micro-magnetic field corresponding to a diameter of the joint is provided,
wherein the control unit comprises:
a sensing part configured to sense a micro-magnetic field output to the antenna band part for a predetermined time;
an amplifying part configured to amplify a sensing value of the sensed micro-magnetic field; and
an RC filter part configured to convert the amplified sensing value so as to adjust the high frequency voltage.

2. The device of claim 1, wherein the first antenna and the second antenna of the antenna band part are respectively formed in a non-linear shape, and are spaced apart from each other.

3. The device of claim 1, wherein the control unit comprises:
a high frequency forming part configured to form the high frequency voltage; and
an output adjusting part configured to adjust the formed high frequency voltage and output the adjusted high frequency voltage to the antenna band part.

4. (canceled)

5. The device of claim 1, wherein the control unit is configured to calculate a ratio of a reference area related to the diameter of the joint on the basis of the converted DC level signal, and is configured to adjust the high frequency voltage on the basis of the calculated ratio.

6. The device of claim 5, wherein the control unit is configured to preset a reference output value that corresponds to a median value for a diameter size of the joint, and is configured to adjust the high frequency voltage on the basis of the preset reference output value and the calculated ratio.

7. The device of claim 6, wherein the control unit is configured to modify a pulse width of the high frequency voltage, thereby generating a constant micro-magnetic field regardless of a size of the joint.

8. The device of claim 7, wherein the control unit is configured to set the reference output value with a duty of 50%, and is configured to reduce the pulse width to be smaller than the 50% duty when a sensing value amplified by the amplifying part is larger than the reference output value and is configured to increase the pulse width to be larger than the 50% duty when the sensing value amplified by the amplifying part is smaller than the reference output value, thereby generating the constant micro-magnetic field.

9. A method for driving a device for supporting height growth by using a pulsed micro-electromagnetic field, the method comprising:
generating, by an antenna part, a micro-magnetic field at a joint in a contactless manner by a first antenna and a second antenna that are provided inside a covering part which surrounds the joint; and
forming, by a control unit, a high frequency voltage and providing the high frequency voltage to the antenna band part and thus generating the micro-magnetic field, and adjusting the generated micro-magnetic field, on the basis of a sensing value of a micro-magnetic field sensed at the second antenna, such that a micro-magnetic field corresponding to a diameter of the joint is provided;
sensing, by a sensing part, a micro-magnetic field output to the antenna band part for a predetermined time;
amplifying, by an amplifying part, a sensing value of the sensed micro-magnetic field; and
converting, by an RC filter part, the amplified sensing value into a DC level signal so as to adjust the high frequency voltage.

10. The method of claim 9, wherein the first antenna and the second antenna of the antenna band part are respectively formed in a non-linear shape, and are spaced apart from each other.

11. The method of claim 9, further comprising:
forming, by a high frequency forming part, the high frequency voltage; and
outputting, by an output adjusting part, performed by adjusting the formed high frequency voltage and outputting the adjusted high frequency voltage to the antenna band part.

12. (canceled)

13. The method of claim 9, further comprising:
calculating a ratio of a reference area related to the diameter of the joint on the basis of the converted DC level signal; and
adjusting the high frequency voltage on the basis of the calculated ratio.

14. The method of claim 13, wherein the adjusting of the high frequency voltage comprises:
presetting a reference output value that corresponds to a median value for a diameter size of the joint; and
adjusting the high frequency voltage on the basis of the preset reference output value and the calculated ratio.

15. The method of claim 14, wherein, in the adjusting of the high frequency voltage, a pulse width of the high frequency voltage is modified, thereby generating a constant micro-magnetic field regardless of a size of the joint.

16. The method of claim 15, wherein, in the adjusting of the high frequency voltage, the reference output value is set with a duty of 50%, and the pulse width is reduced to be smaller than the 50% duty when a sensing value amplified by the amplifying part is larger than the reference output value and the pulse width is increased to be larger than the 50% duty when the sensing value amplified by the amplifying part is smaller than the reference output value, thereby generating the constant micro-magnetic field.
